SEQUENCE LISTING (1 sequence)

SEQ ID NO: 1 - protein, 15 aa, artificial sequence
  synthetic sequence
FAKALKALLKALKAL

(19) United States
(12) Patent Application Publication
(10) Pub. No.: US 2007/0207112 A1
(43) Pub. Date: Sep. 6, 2007

(54) Anti-acne composition

(75) Inventors: John L. Gormley, Midland Park, NJ (US); George P. Majewski, Redondo Beach, CA (US); Amit R. Shah, Commack, NY (US)

Correspondence Address: K.F. ROSS P.C., 5683 RIVERDALE AVENUE, SUITE 203 BOX 900, BRONX, NY, 10471-0900, US

(73) Assignee: GRANT INDUSTRIES, INC.

(21) Appl. No.: 11/634,368

(22) Filed: Dec. 5, 2006

Related U.S. Application Data
(60) Provisional application No. 60/778,224, filed on Mar. 2, 2006.

(51) Int. Cl. A61K 8/81; A61K 8/64; A61K 38/10; A61K 8/65; A61K 31/60

(52) U.S. Cl. 424/70.14; 514/14; 514/159; 424/70.16

(57) ABSTRACT

The present invention relates to a composition for treating acne comprising an anti-microbial peptide made from the amino acids phenylalanine, alanine, leucine and lysine with said peptide admixed with salicylic acid and/or salicylate salts in a cosmetic delivery vehicle containing a rehydrating polymer. The invention further relates to a method for treating acne by topically administering one of the compositions in an amount therapeutically effective to reduce the redness and blemishes associated with acne.

### CROSS REFERENCE TO RELATED APPLICATIONS

This application is related to co-pending provisional application Ser. No. 60/748,224 filed 7 Dec. 2005.

### FIELD OF THE INVENTION

This invention relates to topical compositions and methods for treating acne while conditioning facial skin. More specifically, the present invention relates to compositions and methods for the treatment of acne with a topical composition containing a vegetable-derived anti-microbial oligopeptide, based on the amino acids phenylalanine, alanine, leucine and lysine, with salicyclic acid or salicylate salts in a cosmetic delivery vehicle containing a non-comedogenic, rehydrating polymer for improved active delivery and skin moisturization. The cosmetic vehicle may be an emulsion that contains other beneficial anti-acne ingredients, such as natural products, botanical extracts, esters of retinoic acid and oil-absorbing compounds that improve the appearance and health of facial skin.

### BACKGROUND OF THE INVENTION

Acne vulgaris is a skin disorder that inflicts about 85% of people to some degree in their adolescent lives. In severe cases, it can cause facial and emotional scarring. It will continue into adulthood for 18% of the population, with 6% more females suffering chronic outbreaks then males.

Acne is thought to be the result of several environmental and pathophysiological factors like diet, pollution, stress level, androgen (hormone) level and age. External factors contribute to acne, such as friction (acne mechanica) or contact with irritant oils or cosmetics (acne cosmetica). The acne lesion or comedone, is an enlarged hair follicle plugged with oil and bacteria. Blackheads (open comedones) and whiteheads (closed comedones) result from such blockage.

Pilosebaceous units are comprised of a sebaceous (oil producing) gland connected to a hair follicle. Blockage of the pilosebaceous unit is a contributing factor in acne. This can occur from hyperkeratinization from abnormal desquamation of follicular epithelium. In normal skin, sebum is produced which flows unimpeded onto the skin surface through the open follicle. Increased sebum production is also implicated in the development of acne. Sebum is a lipid-rich secretion from sebaceous glands and their size and production volume is under the control of androgenic hormones. The onset of acne is typically associated with hormonal surges before and during puberty.

Another prime factor in acne development is the proliferation of bacterium like Propionbacterium acnes (P. acnes), which generate inflammation and infection. P. acnes is an organism that can multiply in a clogged pilosebaceous unit once an anaerobic environment is created. This leads to pustule formation and an inflammatory response (possibly due to bacterial release of enzymatic and chemical agents that promote inflammation). Other micro-organisms besides P. acnes have been identified and associated with acne, thus requiring an antimicrobial treatment to have broad spectrum activity.

Oral antibiotics are used for treating moderate to severe acne lesions. Long-term use requires routine laboratory monitoring. P. Acne and other bacteria has becomes resistant to many antibiotic and such use is on the decline. Attempts to decrease sebum production using oral vitamin or hormonal regulation can be very effective. Oral Vitamin A acid derivatives are only approved for severe nodular acne but commonly exhibit serious side effects or adverse reactions, including birth defects, which requires careful screening, registration and monitoring for treatment candidates and users.

Numerous topical treatments for acne are available as OTC drugs as a serums (ex. aqueous or hydroalcoholic solution), gels, and emulsion (cream or lotion). These preparations can be effective in varying degrees, but none have been proven to be uniformly effective and without side effects. Treating compositions have included benzoyl peroxide, retinoic acids or retinoid derivatives, antibiotic combinations including clindamycin, tetracycline, doxycycline, or erythromycin with or without benzoyl peroxide, resorcinol, sulfur, azeleic acid, pantothenic acid, retinoic acids and their derivatives have been used in acne treatments as a cocktail of combined antibiotics, vitamins and hormonal regulators.

Benzoyl peroxide can leave the skin with a dry, spotty, mottled appearance and can cause bleach damage to clothing or linens in contact with the face during sleeping or other activities. Topical antibiotics decrease the number of mild to moderate inflammatory lesions by inhibiting the growth of p. acnes and are also associated with skin irritation, dryness, and potential antibiotic resistance. There is still a high demand for effective topical remedies, without side effects.

Comedolytic agents like salicylic acid, glycolic acid and the salts of both acids are popular for exfoliating dead skin cells and opening and draining the pores, but significant irritation can occur owing in part to the relatively high use levels employed in many instances. There still exists the need to effectively use these agents near their lower use limits as opposed to their upper limits in terms of concentrations to avoid dryness or irritation issues. For example, salicylic acid is used at a minimum concentration of 0.5% in the FDA monograph. 21 CFR 333.310 for OTC acne treatments.

XMP.629 is a peptide compound derived from the bactericidal/permeability-increasing protein (BPI), a human host defense protein with low MIC values against P. acnes and a host of other bacterium typically found on the skin (U.S. application 20050148495A1, Lambert et al). No mention, examples or claims are presented connecting the use of salicylic acid in combination with peptide XMP.629. U.S. Pat. No. 6,875,744 B2 specifies short bioactive antimicrobial peptides like FAKALKALLKALKAL-NH₂ (Compound A) that are not based on a human protein lineage and are made exclusively from phenylalanine, alanine, leucine and lysine. Such peptides have been reported to offer a low in-vitro minimum inhibitory concentration (MIC) against P. acnes. These peptides have no comedolytic activity and the peptide HB64 has been reported to be compatible with salicylic acid.

Skin tissue after an acne flare up can be darkened in comparison to the rest of the unaffected skin. Benzoyl peroxide can lighten blemishes by oxygen bleaching action but the same mechanism can stain associated clothing and cause uneven lightening. Botanical ingredients like Rice Bran extract have very mild skin lightening activity. Manuka honey, melaluca oil/extract and Boswellia extract have mild antiseptic and antimicrobial activity. Such natural extracts are generally considered skin beneficial.

Film-forming polymers are generally avoided in anti-acne preparations because of concern about comedogenicity from polymer components clogging pores or preventing exfoliation of dead skin cells. However, such polymers are able to offer controlled delivery of actives and hydrate the skin for extended periods of time. Therefore, a non-occlusive, noncomedogenic polymer that is able to keep the skin moist and maintain skin contact of salicylic acid and peptide is desirable for anti-acne treatment.

There still remains an unmet need for improved anti-acne compositions based on antimicrobial peptide sequences that are synergistic with a comedolytic agent like salicyclic acid, and contain ingredients to lighten acne scars, and are delivered by rehydrating polymers that are not comedogenic or occlusive and maintain the skin in a moist healthy state.

### OBJECTS OF THE INVENTION

It is an object of the invention to provide a new composition for the treatment of acne that is able to keep the skin moist and at the same time to deliver both an antibacterial treatment and a comedolytic effect on the skin for exfoliating dead skin cells and opening and draining the pores.

It is a further object of the invention to provide an improved composition and a method for treating acne based on antimicrobial peptide sequences that are synergistically effective with a comedolytic agent like salicylic acid against the bacteria that cause acne, and that contain ingredients to lighten acne scars, and that are not comedogenic or occlusive to maintain the skin in a moist healthy state.

### SUMMARY OF THE INVENTION

An anti-acne composition is comprised of:

0.001% to about 0.1% by weight percent of a synthetic vegetable-derived anti-microbial short chain peptide of 10-20 amino acid units of primarily phenylalanine, alanine, leucine and lysine;

0.5% to about 2% by weight of salicylic acid, preferably 0.5% to 1% by weight salicylic acid and alkali metal salts thereof; and

the remaining portion forming a cosmetically acceptable delivery vehicle, comprising an aqueous or hydro-alcoholic phase containing 0.01% to about 2% by weight of a non-comedogenic, hydrated film-forming copolymer formed from the solution polymerization of dimethylacrylamide, acrylic acid, polystyrene, and methacrylate monomers.

A preferred feature of the invention is an anti-acne composition comprised of:

0.001% to about 0.11% by weight percent of a synthetic vegetable-derived anti-microbial short chain peptide prepared primarily from the amino acids phenylalanine, alanine, leucine and lysine;

0.5% about 2% by weight of salicylic acid, preferably 0.5% to 1% by weight of salicylic acid and alkali metal salts thereof; and

the remaining portion forming a cosmetically acceptable delivery vehicle, comprising an aqueous phase wherein the aqueous phases contains 0.01% to about 2% by weight of a non-comedogenic, hydrated film-forming copolymer formed from the solution polymerization of dimethylacrylamide, acrylic acid, polystyrene, and methacrylate monomers and an oil phase, where either water or oil may be the a continuous phase to form an emulsified delivery vehicle.

The aqueous component of the cosmetically acceptable delivery vehicle contains. 0.1-40% by weight of one or more skin beneficial natural products and botanical extracts with mild skin lightening and/or anti-microbial action, such natural products include rice-bran extract, manuka honey or boswellia extract.

Where the delivery vehicle is an emulsion, the oil-phase of the emulsion contains one or more of the following ingredients: 1.0 to about 20% by weight of an oil-absorbing silicone-elastomer crosspolymer gel and 0% to about 0.5% by weight of a retinoic acid ester.

Preferably the short chain peptide comprises 15 to 20 amino acids and comprises at least 95% phenylalanine, alanine, leucine and lysine as the amino acids and at its C terminal has either a free carboxy group or a free carboxamido group, most preferably a free carboxamido group. More preferably the short chain peptide comprises 100% phenylalanine, alanine, leucine and lysine as the amino acids and is 15 amino acids in length. The most preferred short chain peptide is Compound A having the formula: FAKALKALLKALKAL-NH₂;

The aqueous component of the cosmetically acceptable delivery vehicle contains 0.1-40% by weight of one or more skin beneficial natural products and botanical extracts with mild skin lightening and/or anti-microbial action, such natural products include rice-bran extract, manuka honey or boswellia extract.

Where the delivery vehicle is an emulsion, the oil-phase of the emulsion contains one or more of the following ingredients: 1.0 to about 20% by weight of an oil-absorbing silicone-elastomer crosspolymer gel and 0% to about 0.5% by weight of a retinoic acid ester.

Preferably the short chain peptide comprises 15 to 20 amino acids and comprises at least 95% phenylalanine, alanine, leucine and lysine as the amino acids and at its C terminal has either a free carboxy group or a free carboxamido group, most preferably a free carboxamido group. More preferably the short chain peptide comprises 100% phenylalanine, alanine, leucine and lysine as the amino acids and is 15 amino acids in length. The most preferred short chain peptide is Compound A having the formula: FAKALKALLKALKAL-NH₂;

The short chain peptides are not new compounds and are described in U.S. Pat. No. 6,875,744 B2. The peptide that is designated as Compound A is also described throughout this application as SEQ ID NO: 1 according to the following:

### Phe Ala Lys Ala Leu Lys Ala Leu Leu Lys Ala Leu Lys Ala Leu

As will be described more fully, the combination of the preferred peptide sequence and sodium salicylate is synergistic towards killing P. Acne bacteria. Salicylic acid is USP grade and preferably at or about 0.5% by weight, which is the minimum quantity defined as acceptable for OTC acne treatments containing salicylic acid. The delivery vehicle can be deionized water with a USP acceptable preservative system, or a mixture of water and lower alkyl alcohol, preferably ethyl or isopropyl alcohol. In one aspect, the alcohol level is substantially high enough to be self-preserving or around 15% by weight, but otherwise limited in concentration to avoid drying the skin. In other aspects, the alcohol level is less than 15% and a USP preservative is used in the system.

The aqueous component of the cosmetic delivery vehicle contains 0.1% to about 2% by weight of a non-comedogenic, hydrated film-forming copolymer formed from the solution polymerization of dimethylacrylamide, acrylic acid, polystyrene, and methacrylate monomers and having a viscosity of 50,000 to 300,000 cps at 15% solids in water. A preferred film-forming polymer is InvisaSkin™ RB (Grant Industries, Elmwood Park N.J. USA), a hydrated dimethylacrylamide/acrylic acid/polystyrene/methacrylate copolymer mixed with Oryza sativa (Rice) bran extract and is a liquid with a viscosity fo 80,000 cps with a non-volatile content of about 13.0 to about 17.0% by weight polymer. The aforementioned polymer was originally prepared as a non-comedogenic surgical aid for adhering skin/mucal membranes and has beneficial adhesion and hydration properties which are advantageous in acne treatments requiring a liquid polymer.

Optional ingredients may be included as part of the delivery vehicle. These include, but are not limited to, mild surfactants like C₁₂-C₁₆ alkylpolyglucosides, ethoxylated fatty alcohols, preferably laureth-4, laureth-23; non-film forming polymeric stabilizers like acrylate or acrylamide copolymers; humectants and polyols like glycerin, propylene glycol or butylene glycol(s); and botanical extracts as will be delineated in more detail by way of example. Benzoyl peroxide is specifically excluded due to the potential to oxidize the peptide and cause a loss of antimicrobial properties.

The aqueous component of the cosmetically acceptable delivery vehicle may contain 0.1 to about 40% by weight of one or more skin beneficial natural products and botanical extracts with mild skin lightening and/or anti-microbial action, such natural products preferably originating from rice-bran extract, manuka honey or boswellia extract, wherein, such ingredients are used as obtained in the crude state or preferably used after being converted and filtered following a partial yeast or enzyme digestion period.

Additional surfactants or emulsifiers are required to stabilize the mixed oil and water phases. Many commercial choices of surfactant types are available per the McCutcheons emulsifiers guide book and are acceptable herein if they are non-irritating to the skin. Preferred emulsifiers for the oil phase are C₁₆-C₂₆ alkylpolyglucosides, fatty alcohol ethoxylates, including but not, limited to laureth-4, Oleth-2 or a broad spectrum of hydrogenated castor oil ethoxylates.

Where the delivery vehicle is an emulsion, the oil-phase of the emulsion contains one or more of the following ingredients: 1.0 to about 20% by weight of a sebum absorbing agent, preferably a silicone-elastomer cross-polymer gel and 0.01% to about 0.5% by weight of a retinoic acid ester.

More specifically, the silicone elastomer gel may be selected from the group consisting of the reaction product of:

≡Si—H containing polysiloxane with:

an alpha, omega diene, preferably an alpha, omega vinylpolydimethicone, in the presence of;

platinum catalyst and;

solvent comprised of low molecular weight polysiloxane (linear or cyclic), vegetable oils (like jojoba or castor), paraffin, petrolatum, hydrogenated polyisobutene, and mineral oil,

such that the solvent content of the silicone elastomer gel is 85-96% by weight, preferably 90-95% by weight;

wherein, the elastomer gel viscosity is greater than 200 cs, preferably greater than 50,000 cs to about 4,000,000 cs on the high end of viscosity and

wherein, the ≡Si—H containing polysiloxane of part I is represented by compounds of formula: SiO((CH₃)₂SiO)ₐ(CH₃HSiO)_{b}Si(CH₃)₃ or formula H(CH₃)₂SiO((CH₃)₂SiO)ₐSi(CH₃)₂H or formula H(CH₃)₂SiO((CH₃)₂SiO)ₐ(CH₃HSiO)_{b}Si(CH₃)₂H, where a is 1-250 and b is 1-250;

where, the alpha, omega diene of part II is a compound of the formula CH₂═CH(CH₂)ₓCH═CH₂ with representative examples including 1,4-pentadiene; 1,5-hexadiene; 1,6-heptadiene; 1,7-octadiene; 1,8-nonadiene; 1,9-decadiene; 1,11-dodecadiene; 1,13-tetradecadiene and 1,19-eicosadiene or the organo-silicone formula CH₂═CH(CH₃)₂SiO((CH₃)₂SiO)_{c}Si(CH₃)₂CH═CH₂ where c is 1-200;

where the platinum catalyst is represented by hexachloroplatinic acid in a solvent, or a platinum (0) complex of Pt₂{[(CH₂═CH)Me₂Si]₂O)}.

Examples of silicone elastomer gels include:

a cross-linked or partially cross-linked cyclomethicone (and) dimethicone crosspolymer

a cross-linked or partially cross-linked polydimethicone crosspolymer (For example INCI name polysilicone-11, more specifically Gransil GCM-5 a gel with D5 cyclomethicone as solvent, from Grant Industries, Elmwood Park N.J.);

a cross-linked or partially cross-linked cyclomethicone (and) vinyldimethicone/methicone crosspolymer

a cross-linked dimethicone/vinyldimethicone Crosspolymer.

Other sebum absorption or sebum inhibiting agents useful in this invention are laponite,

bentone clays, silica, magnesium aluminum silicate, and flax linseed extract (for metabolic down regulation of sebum).

The preferred ester of retinoic acid is: all-trans retinoic 1-hydroxy-3,3-dimethyl-2-butanone ester Formula 1

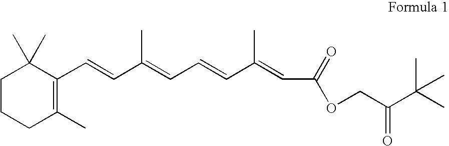

Other nonessential ingredients in the serum or emulsion may include one or more of the following classes of ingredients:

perfumes and essential oils;

sun filters or sunscreen materials, including chemical sunscreens and dispersed physical sunscreens, including those based on titanium dioxide or zinc oxide;

vitamins and their precursors like retinyl palmitate or acetate, Vitamin B as panthenol and its derivatives, Vitamin E as tocopheryl acetate, Vitamin F as polyunsaturated fatty acid esters such as gamma-linolenic acid esters;

skin care agents, such as ceramides either as natural materials or functional mimics of natural ceramides, phospholipids cholesterol; and phytosphingosines;

dispersed inorganics and pigments, including fumed silica, microfine pigments, particularly oxides and silicates, e.g. iron oxide, particularly coated iron oxides, and/or titanium dioxide, and ceramic materials such as boron nitride, or other solid components such as barium sulfate, polydimethylsilsequioxane or nylon particles of around 4 microns average particle size;

proteins and peptides that are not anti-microbial in nature;

polymeric stabilizers like acrylate and acrylamide copolymers, preferred Granthix APP (Grant Industries) or Simulgel EG (Seppic) can be employed to help stabilize the emulsion.

### EXAMPLES

### Example 1

In-Vitro testing for the synergetic inhibitory effects of Compound A and salicylic acid on the growth of P. acnes.

The objective of this experiment was to establish the minimal inhibitory concentration (MIC) of the peptide (Compound A) towards the bacterium P. acnes, in the presence of a range of concentrations of sodium salt of salicylic acid. MIC is defined as the lowest concentration of an active material resulting in lack of microbial growth. P. acnes (BD, cat. #237038, batch #6152097) was inoculated at 3 different dilutions and grown on blood agar plates for one week in anaerobic condition at 37 deg. C. Bacteria were then inoculated in thioglycollate broth in a 96 well plate, at the density equal to 0.5 McFarland standard in the presence of peptide (Compound A) concentrations ranging from 0.25 ug/ml to 32 ug/ml and salicylate concentrations ranging from 0.08% to 5% (checker method). MIC was determined visually and confirmed by spectrophotometry at 490 nm and 590 nm on the BioRad 3550-UV microplate reader.

The results show that addition of sodium salicylate to the peptide (Compound A) preparations strongly enhance its antimicrobial activity. Growing concentrations of salicylate caused Compound A's MIC to decrease in a dose-dependant manner, as shown on Table 1.

| Na salicylate | MIC for Compound A | |
|---|---|---|
| (%) | (ug/ml) | |
| 0 | 8 | ug/ml |
| 0.08 | 2 | ug/ml |
| 0.16 | 0.5 | ug/ml |
| 0.32 | 0.5 | ug/ml |
| 0.64 | 0.25 | ug/ml |
| 1.25 and up | <0.25 | ug/ml |

The addition of about 0.5% by weight salicylate to Compound A preparations unexpectedly decreased the amount of peptide necessary for achieving desired anti-acne effect in the finished product.

The comedolytic agent salicylic acid does not posses any antibacterial activity against P. acnes (U.S. Pat. No. 4,520,133 Dines, et al.).

### Examples 2-5

### Formulation Examples

| | | Example 2 | Example 3 | Example 4 | Example 5 |
|---|---|---|---|---|---|
| | Ingredient | K-7825 | K-7826 | K-7827 | G101-250-6 |
| Phase | Ex. Code | Control | Inventive | Inventive | Inventive |
| (1) | Deionized water | q.s to 100% | q.s to 100% | q.s to | q.s to 100% |
| | | | | 100% | |
| | Oryza sativa (Rice) | 2.00 | 2.00 | 2.00 | 2.00 |
| | bran extract¹ | | | | |
| | Manuka honey¹ | 2.00 | 2.00 | 2.00 | 2.00 |
| | Bosweli extract¹ | 2.00 | 2.00 | 2.00 | 2.00 |
| | 1-3,Butylene Glycol | 3.00 | 3.00 | 3.00 | 3.00 |
| | Granthix APP² | 3.00 | 3.00 | 3.00 | 3.00 |
| | Simulgel EG³ | 4.00 | 4.00 | 4.00 | 4.00 |
| (2) | dimethylacrylamide, | 0.75 | 0.75 | 0.75 | 0.75 |
| | acrylic acid, polystyrene, | | | | |
| | methacrylate | | | | |
| | copolymer⁴ | | | | |
| | HB 64 Peptide | — | 0.010 | 0.010 | 0.001 |
| (3) | SDA-40 Alcohol | 20.00 | 20.00 | 20.00 | 20.00 |
| | Salicylic Acid | 2.00 | 2.00 | 0.50 | 2.00 |

¹BC Research Company, Elmwood Park N.J.,
²Isohexadecane (and) Ammonium Polyacryloyldimethyl Taurate (and) Polysorbate 80,
³Seppic Inc.
⁴InvisaSkin ™ Polymer, Grant Industries, Elmwood Park N.J.

Procedure: Weigh Phase 1 and homogenize. Weigh phase-2 and add to phase-1 with stirring. Mix phase-3 and add to stirring phase 1-2.

### Example 6

### Application of Formulation Examples

| | | Example 2 (K-7825) | | | | | | | Example 3 (K-7826) 2% | | | | | | |
|---|---|---|---|---|---|---|---|---|---|---|---|---|---|---|---|
| | | 2% Salicylic acid, no peptide | | | | | | | Salicylic Acid + 0.01% Peptide | | | | | | |
| | | Day of study | | | | | | | Day of Study | | | | | | |
| Facial Lesions | Sub. No. | 0 | 3 | 7 | 14 | 21 | 28 | 35 | 0 | 3 | 7 | 14 | 21 | 28 | 35 |
| Total | 1 | 60 | 44 | 39 | 25 | 31 | 28 | 34 | 80 | 64 | 22 | 11 | 19 | 11 | 8 |
| | 2 | 44 | 43 | 13 | 5 | 19 | 14 | 10 | 33 | 28 | 15 | 3 | 8 | 2 | 0 |
| | Mean | 52 | 44 | 26 | 15 | 25 | 21 | 22 | 57 | 46 | 19 | 7 | 14 | 7 | 4 |
| | % | | 15 | 50 | 71 | 52 | 60 | 58 | | 19 | 67 | 88 | 75 | 88 | 93 |
| | Difference | | | | | | | | | | | | | | |

### Example 7

### Application of Formulation Examples

| | | Example 2 (K-7825) | | | | | | | Example 4 (K-7827) 0.5% | | | | | | |
|---|---|---|---|---|---|---|---|---|---|---|---|---|---|---|---|
| | | 2% Salicylic acid, no peptide | | | | | | | Salicylic Acid + 0.01% Peptide | | | | | | |
| Facial | | Day of Study | | | | | | | Day of Study | | | | | | |
| Lesions | Sub. No. | 0 | 3 | 7 | 14 | 21 | 28 | 35 | 0 | 3 | 7 | 14 | 21 | 28 | 35 |
| Total | 1 | 58 | 47 | 34 | 28 | 34 | 28 | 26 | 62 | 53 | 21 | 13 | 18 | 16 | 10 |
| | 2 | 28 | 30 | 10 | 10 | 22 | 17 | 40 | 24 | 24 | 19 | 9 | 16 | 8 | 33 |
| | Mean | 43 | 39 | 22 | 19 | 28 | 23 | 33 | 43 | 39 | 20 | 11 | 17 | 12 | 22 |
| | % | | 9 | 49 | 56 | 35 | 47 | 23 | | 9 | 53 | 74 | 60 | 72 | 49 |
| | Difference | | | | | | | | | | | | | | |

Examples 6 and 7 include ex-vivo results using split-face application of a control with 2% by weight salicylic acid versus the inventive composition with 0.01% by weight peptide (Compound A) at two levels of salicyclic acid (2% and 0.5% by weight, respectively). In all cases, the addition of peptide led to a reduction of acne compared to the control. Formula K2827 in example 7 employed 75% less salicylic acid and exhibited improved anti-acne performance compared to the control composition. No irritation occurred on any test containing the rehydrating copolymer.

### Example 8

Alcohol-free emulsion

| | | Example 8 |
|---|---|---|
| Phase | Ingredient | Weight % |
| (1) | Deionized water | q.s. |
| | C_{10–12} alkyl polyglucoside | 0.12 |
| | 1,3-Butylene Glycol | 4.00 |
| | Phenoxyethanol | 0.10 |
| | Polysorbate-20 | 0.40 |
| | Peptide HB64 | 0.01 |
| | Salicylic Acid | 0.50 |
| | dimethylacrylamide, acrylic acid, | 0.75 |
| | polystyrene, methacrylate copolymer | |
| (2) | Cyclomethicone and Polysilicone-11⁵ | 75.00 |
| | Laureth-4 | 1.00 |
| | Simulgel-EG | 0.70 |
| | 5 cst polydimethlysiloxane | 7.80 |
| | all-trans retinoic 1-hydroxy-3,3- | 0.15 |
| | dimethyl-2-butanone ester | |

⁵Gransil-GCM5 Grant Industries, Elmwood Park N.J.

Procedure:

Weigh Part 1 in main kettle equipped with side sweep agitation, mix @ 150-200 RPM for 15 minutes. Weigh Part 2 In side kettle equipped with homogenizer. Mix until homogeneous. Slowly transfer Part 2 to Part 1 while mixing.

1. A composition for the topical treatment of acne comprising:
0.001% to about 0.1% by weight percent of a synthetic vegetable-derived anti-microbial short chain peptide of 10-20 amino acid units of primarily phenylalanine, alanine, leucine and lysine,
0.5% to about 2% by weight of salicylic acid and alkali metal salts thereof;
97.9% to about 99.4% by weight aqueous delivery vehicle containing 0.01% to about 2% by weight of a non-comedogenic, hydrated film-forming copolymer formed from the solution polymerization of dimethylacrylamide, acrylic acid, polystyrene, and methacrylate monomers.

2. The composition of claim 1 where the peptide is 15 amino acid units of phenylalanine, alanine, leucine and lysine with the sequence FAKALKALLKALKAL-NH₂.

3. The composition of claim 1 where the delivery vehicle contains 10% to about 20% C₁-C₃ alkyl alcohols.

4. The composition of claim 1 with 0.5% to 1% by weight salicylic acid and alkali metal salts thereof.

5. The composition of claim 1 where the peptide weight percent is 0.005%>to about 0.2%.

6. The composition of claim 1 wherein the aqueous delivery system contains 0.1 to about 40% by weight of natural products originating from rice-bran extract, manuka honey melaluca oil/extract or boswellia extract.

7. The composition of claim 1 in which the aqueous delivery vehicle forms an emulsion with an oil, and in, which either water or oil is the continuous phase, wherein the aqueous component contains 0.0104 to about 2% by weight of the non-comedogenic, hydrated film-forming copolymer formed from the solution polymerization of dimethylacrylamide, acrylic acid, polystyrene, and methacrylate monomers.

8. The composition of claim 7 where the oil phase contains 1% to 78% by weight of an oil-absorbing silicone-elastomer crosspolymer gel.

9. The composition of claim 7 where the oil phase contains 0.01 to about 0.5% by weight of an ester of retinoic acid.

10. The composition of claim 8 where the silicone-elastomer crosspolymer gel contains polysilicone-11.

11. (canceled)

12. The composition of claim 9 where the ester of retinoic acid is all-trans retinoic 1-hydroxy-3,3-dimethyl-2-butanone ester.

13. The composition of claim 3 wherein the film-forming polymer is a hydrated dimethylacrylamide/acrylic acid/polystyrene/methacrylate copolymer mixed with Oryza sativa bran extract with a non-volatiles content of about 13.0 to about 17.0% by weight.

14. The composition of claim 7 wherein the film-forming polymer is a hydrated dimethylacrylamide/acrylic acid/polystyrene/methacrylate copolymer mixed with Oryza sativa bran extract with a non-volatiles content of about 13.0 to about 17.0% by weight.

15. A method of applying to the skin of a patient suffering from acne, the composition for the topical treatment of acne defined in claim 1 which comprises the step of topically administering to the skin of said patient a therapeutically effective amount of said composition.

16. The method of claim 15, wherein the composition for the treatment of acne comprises the peptide that is Compound A, namely, FAFALKALLKALKAL-NH₂.

17. The method of claim 15, wherein the delivery vehicle in the composition for the topical treatment of acne contains 1% to about 20% C₂-C₃ alkyl alcohols.

18. The method of claim 15, wherein the composition for the topical treatment of acne contains 0.5% to 1% by weight salicylic acid or an alkali metal salt thereof.

19. The method of claim 18, wherein the peptide in the composition for the topical treatment of acne is present in an amount of 0.005% to about 0.2% weight-percent.

20. The method of claim 15, wherein the aqueous delivery vehicle in the composition for the topical treatment of acne contains 0.1 to about 40% by weight of natural products originating from rice-bran extract, manuka honey, melaluca oil/extract or boswellia extract.

21. The method of claim 15 wherein the film-forming polymer in the composition for the topical treatment of acne is a hydrated dimethylacrylamide/acrylic acid/polystyrene/methacrylate copolymer mixed with Oryza sativa bran extract with a non-volatiles content of about 13.0 to about 17.0% by weight.

22. A method of applying to the skin of a patient suffering from acne, the composition for the topical treatment of acne defined in claim 7 which comprises the step of topically administering to the skin of said patient a therapeutically effective amount of said composition.

23. The method of claim 22, wherein the oil phase contains 1% to 78% by weight of an oil-absorbing silicone-elastomer crosspolymer gel.

24. The method of claim 23, wherein the oil phase contains 0.01 to about 0.5% by weight of an ester of retinoic acid.

25. The method of claim 23, wherein the silicone-elastomer crosspolymer gel contains polysilicone-11.

26. (canceled)

27. The method of claim 24, wherein the ester of retinoic acid is all-trans retinoic 1-hydroxy-3,3-dimethyl-2-butanone ester.

28. The method of claim 22 wherein the film-forming polymer in the composition for the topical treatment of acne is a hydrated dimethylacrylamide/acrylic acid/polystyrene/methacrylate copolymer mixed with Oryza sativa bran extract with a non-volatiles content of about 13.0 to about 17.0% by weight.

29. The method of claim 15, wherein the composition for the topical treatment of ache is presented in a patch, gel stick, spray, aerosol, wipe or swab.

30. The method of claim 22, wherein the composition for the topical treatment of acne is presented in a patch, gel stick, spray, aerosol, wipe or swab.
